(19) United States
(12) Patent Application Publication
(10) Pub. No.: US 2024/0426823 A1
(43) Pub. Date: Dec. 26, 2024

(54) METHODS AND COMPOSITIONS FOR TREATING TRIPLE NEGATIVE BREAST CANCER (TNBC)

(71) Applicants: INSTITUT NATIONAL DE LA SANTÉ ET DE LA RECHERCHE MÉDICALE, Paris (FR); UNIVERSITÉ DE MONTPELLIER, Montpellier (FR); INSTITUT RÉGIONAL DU CANCER DE MONTPELLIER, Montpellier (FR); CENTRE HOSPITALIER UNIVERSITAIRE DE NIMES, Nimes (FR)

(72) Inventors: Emmanuelle LIAUDET-COOPMAN, Montpellier (FR); Lindsay ALCARAZ-CACCHIA, Montpellier (FR); Pascal ROGER, Montepllier (FR); Séverine GUIU, Montpellier (FR)

(21) Appl. No.: 18/706,618

(22) PCT Filed: Nov. 2, 2022
(86) PCT No.: PCT/EP2022/080508

§ 371 Date: May 1, 2024

(30) Foreign Application Priority Data
Nov. 3, 2021 (EP) 21306545.1

(51) Int. Cl. G01N 33/574

(52) U.S. Cl. CPC G01N 33/57415; G01N 2800/52

(57) ABSTRACT

Inventors analyzed the prognostic value of tumor and stromal-derived SPARC in a large series that included 148 non-metastatic TNBC patients with a long follow-up by immunohistochemistry. They show that SPARC expression was detected in cancer cells (42.4%), cancer-associated fibroblasts (CATs) (88.1%). TAMs (77.1%), endothelial cells (75.2%) and TILs (9.8%). Recurrence-free survival (RFS) was significantly lower for patients with a positive expression of SPARC in CATs (SPARC+CATs) with a median follow-up of 5.4 years. SPARC expression in CATs was found to be an independent prognostic factor in multivariate analysis. Accordingly, the present invention relates to a method for predicting the survival time of a subject suffering from triple-negative breast cancer (TNBC) comprising determining the expression level of Secreted Protein Acidic and Rich in Cysteine (SPARC) in cancer-associated fibroblasts (CATs) in a biological sample obtained from the subject wherein said positive expression of SPARC in CATs (SPARC+CAFs) correlates with a short survival time of the subject.

### FIELD OF THE INVENTION

The invention is in the field of oncology, more particularly the invention relates to a method and compositions for treating triple negative breast cancer (TNBC).

### BACKGROUND OF THE INVENTION

Triple-negative breast cancers (TNBC) are defined by the lack of estrogen receptor (ER), progesterone receptor (PR) and HER2 expression/amplification. TNBC represent 15% of all breast cancers [1]. At present, chemotherapy is the mainstay of treatment for early-stage and advanced TNBC. Despite surgery, adjuvant chemotherapy and radiotherapy, prognosis in patients with TNBC is poor, mainly due to the disease heterogeneity and the lack of specific targets and targeted therapeutics. TNBC is characterized with unique tumor microenvironment, which differs from other subtypes, and associated with induction of proliferation, angiogenesis, inhibition of apoptosis, immune suppression, and drug resistance [2]. The components of the tumor microenvironment including transformed extracellular matrix, soluble factors, immune cells, and re-programmed fibroblasts together hamper antitumor response and helps progression and metastasis of TNBC. Stroma heterogeneity in TNBC remains poorly understood, limiting the development of stromal-targeted therapies.

The matricellular protein SPARC (Secreted Protein Acidic and Rich in Cysteine), also known as osteonectin or basement membrane 40 (BM40), is a Ca2+-binding glycoprotein that regulates extracellular matrix assembly and deposition, growth factor signaling, and interactions between cells and their surrounding stroma [3-6]. In cancer, SPARC is mainly secreted by the neighboring stroma, but also by cancer cells [7-9]. SPARC plays oncogenic or tumor-suppressive roles depending of the cancer type [10, 11]. In breast cancer, SPARC has a pro-tumorigenic role and has been associated with worse prognosis [8, 12-17]; however, some studies also reported its anti-tumorigenic functions [18-20].

### SUMMARY OF THE INVENTION

The present invention relates to a method for predicting the survival time of a subject suffering from triple-negative breast cancer (TNBC) comprising determining the expression level of the expression of Secreted Protein Acidic and Rich in Cysteine (SPARC) in cancer-associated fibroblasts (CAFs) in a biological sample obtained from the subject wherein said positive expression of SPARC in CAFs correlates with a short survival time of the subject. In particular, the invention is defined by the claims.

### DETAILED DESCRIPTION OF THE INVENTION

Inventors analyzed the prognostic value of tumor and stromal-derived SPARC in a large series that included 148 non-metastatic TNBC patients with a long follow-up by immunohistochemistry. They show that SPARC expression was detected in cancer cells (42.4%), cancer-associated fibroblasts (CAFs) (88.1%), TAMs (77.1%), endothelial cells (75.2%) and TILs (9.8%). Recurrence-free survival (RFS) was significantly lower for patients with a positive expression of SPARC in CAFs (SPARC+ CAFs) with a median follow-up of 5.4 years. SPARC expression in CAFs was found to be an independent prognostic factor in multivariate analysis. Tumor and stromal SPARC was observed in TNBC cytosols, patient-tumor derived xenografts (PDXs), and in cell lines. SPARC was expressed by different subsets of CAFs including myofibroblasts and inflammatory CAFs. Fibroblast-derived SPARC inhibited cell adhesion and stimulated migration, and invasion of TNBC cells.

Accordingly, in a first aspect, the invention relates to a method for predicting the survival time of a subject suffering from triple-negative breast cancer (TNBC) comprising determining the expression of Secreted Protein Acidic and Rich in Cysteine (SPARC) in cancer-associated fibroblasts (CAFs) in a biological sample obtained from the subject wherein said positive expression of SPARC in CAFs correlates with a short survival time of the subject.

In a particular embodiment, the method according to the invention comprising the steps of i) quantifying the expression of SPARC in CAFs in a biological sample obtained from the subject; ii) comparing the expression quantified at step i) with its predetermined reference value and iii) concluding that the subject will have a short survival time when the expression of SPARC in CAFs is higher than its predetermined reference value or concluding that the subject will have a long survival time when the expression of SPARC in CAFs is lower than its predetermined reference value.

As used herein, the term “predicting” means that the subject to be analyzed by the method of the invention is allocated either into the group of subjects who will relapse, or into a group of subjects who will not relapse after a treatment.

The method is particularly suitable for predicting the duration of the overall survival (OS), progression-free survival (PFS) and/or the disease-free survival (DFS or RFS Recurrence Free Survival or Relapse Free Survival) of the cancer subject. Those of skill in the art will recognize that OS survival time is generally based on and expressed as the percentage of people who survive a certain type of cancer for a specific amount of time. Cancer statistics often use an overall five-year survival rate. In general, OS rates do not specify whether cancer survivors are still undergoing treatment at five years or if they have become cancer-free (achieved remission). DSF gives more specific information and is the number of people with a particular cancer who achieve remission. Also, progression-free survival (PFS) rates (the number of people who still have cancer, but their disease does not progress) include people who may have had some success with treatment, but the cancer has not disappeared completely. As used herein, the expression “short survival time” indicates that the subject will have a survival time that will be lower than the median (or mean) observed in the general population of subjects suffering from said cancer. When the subject will have a short survival time, it is meant that the subject will have a “poor prognosis”. Inversely, the expression “long survival time” indicates that the subject will have a survival time that will be higher than the median (or mean) observed in the general population of subjects suffering from said cancer. When the subject will have a long survival time, it is meant that the subject will have a “good prognosis”.

As used herein, the term “subject” denotes a mammal, such as a rodent, a feline, a canine, and a primate. Particularly, the subject according to the invention is a human. More particularly, the subject according to the invention has or is susceptible to have TNBC.

As used herein, the term «triple-negative breast cancer» refer to a cancer which is defined by the lack of estrogen receptor (ER), progesterone receptor (PR) and HER2 expression/amplification. TNBC represent 15% of all breast cancers. In a particular embodiment, the TNBC is non-metastatic. In another embodiment, the TNBC is metastatic.

As used herein, the term “Secreted Protein Acidic and Rich in Cysteine” (SPARC) refers to a glycoprotein associated with the extracellular matrix which is widely distributed in human tissues during development. It is described as regulating morphogenesis, cell proliferation and differentiation. Although its specific role still remains uncertain, its high degree of conservation between species suggests a high pressure for conserving it during evolution. SPARC has its general meaning in the art and refers to a Ca2+-binding glycoprotein that regulates extracellular matrix assembly and deposition, growth factor signalling, and interactions between cells and their surrounding extracellular matrix. In cancer, SPARC is mainly secreted by the neighbouring stroma, but also by cancer cells and plays an oncogenic or a tumour-suppressive role.

As used herein, the term “cancer-associated fibroblasts” also known as tumour-associated fibroblast; carcinogenic-associated fibroblast; activated fibroblast is a cell type within the tumor microenvironment that promotes tumorigenic features by initiating the remodelling of the extracellular matrix or by secreting cytokines. CAFs are a complex and abundant cell type within the tumour microenvironment; the number cannot decrease, as they are unable to undergo apoptosis.

As used herein, the term “expression” refers to SPARC signal in CAFs. More particularly, SPARC signal in CAFs, TAMs, endothelial cells, and TILs was scored as negative (<50% of stained cells), or positive (≥50% of stained cells).

SPARC signal in normal epithelial breast tissue samples (N) was compared to the paired tumor sample (T) and scored as lower (N<T), equal (=), or higher (N≥T). As shown in the Example, subjects with a positive expression of SPARC in CAFs (SPARC CAFs) had a lower RFS (p=0.034): the 3-year RFS rates were 72% (CI 95% [61.5-79.6]) and 93% (CI 95% [59.1-99.0]) for SPARC CAFs and the other TNBCs, respectively. There was a trend for better RFS in patients with positive expression of SPARC in TAMs (SPARC TAMs) (p=0.088): the 3-year RFS rates were 81% (CI 95% [70.2-87.7]) and 62% (CI 95% [39.2-78.2]) for SPARC TAMs and the other TNBCs, respectively.

Accordingly, in a particular embodiment, the method according to the invention wherein the subject defined as having positive expression of SPARC in CAFs is SPARC CAFs.

Typically the invention relates to a method of predicting the survival time of a subject suffering from triple-negative breast cancer (TNBC) comprising determining the expression of Secreted Protein Acidic and Rich in Cysteine (SPARC) in cancer-associated fibroblasts (CAFs) in a biological sample obtained from the subject wherein said positive expression of SPARC in CAFs (SPARC CAFs) correlates with a short survival time of the subject.

As used herein, the term “biological sample” refers to any sample obtained from a subject, such as a serum sample, a plasma sample, a urine sample, a blood sample, a lymph sample, or a tissue biopsy. In a particular embodiment, biological sample for the determination of an expression level include samples such as a blood sample, a lymph sample, or a biopsy. In the context of the invention, the biological sample is tumor tissue sample.

As used herein, the term “tumor tissue sample” has its general meaning in the art and encompasses pieces or slices of tissue that have been removed including following a surgical tumor resection. The tumor tissue sample can be subjected to a variety of well-known postcollection preparative and storage techniques (e.g., fixation, storage, freezing, etc.) prior to determining the cell densities. Typically the tumor tissue sample is fixed in formalin and embedded in a rigid fixative, such as paraffin (wax) or epoxy, which is placed in a mould and later hardened to produce a block which is readily cut. Thin slices of material can be then prepared using a microtome, placed on a glass slide and submitted e.g. to immunohistochemistry (IHC) (using an IHC automate such as BenchMark® XT or Autostainer Dako, for obtaining stained slides). The tumour tissue sample can be used in microarrays, called as tissue microarrays (TMAs) (see material and methods). TMA consists of paraffin blocks in which up to 1000 separate tissue cores are assembled in array fashion to allow multiplex histological analysis. This technology allows rapid visualization of molecular targets in tissue specimens at a time, either 5 at the DNA, RNA or protein level. TMA technology is described in WO2004000992, U.S. Pat. No. 8,068,988, Olli et al 2001 Human Molecular Genetics, Tzankov et al 2005, Elsevier; Kononen et al 1198; Nature Medicine.

In some embodiments, the expression and/or expression of SPARC is determined by Immunohistochemistry (IHC).

As used herein, the term “predetermined reference value” refers to a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of cell densities in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the expression of SPARC in a group of reference, one can use algorithmic analysis for the statistic treatment of the expression in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER. S AS, DESIGNROC.FOR, MULTIREADER POWE S AS, CREATE-ROC.SAS, GB STAT VIO.O (Dynamic Microsystems, Inc. Silver Spring, Md., USA), etc.

In some embodiments, the predetermined reference value is determined by carrying out a method comprising the steps of a) providing a collection of biological samples from subject suffering from TNBC; b) providing, for each biological sample provided at step a), information relating to the actual clinical outcome for the corresponding subject (i.e. the duration of the disease-free survival (DFS or RFS) and/or the overall survival (OS)); c) providing a serial of arbitrary quantification values; d) determining the expression of SPARC for each biological sample contained in the collection provided at step a); e) classifying said biological sample in two groups for one specific arbitrary quantification value provided at step c), respectively: (i) a first group comprising biological sample that exhibit a quantification value for level that is lower than the said arbitrary quantification value contained in the said serial of quantification values; (ii) a second group comprising biological sample that exhibit a quantification value for said level that is higher than the said arbitrary quantification value contained in the said serial of quantification values; whereby two groups of biological sample are obtained for the said specific quantification value, wherein the biological sample of each group are separately enumerated; f) calculating the statistical significance between (i) the quantification value obtained at step e) and (ii) the actual clinical outcome of the subjects from which biological sample contained in the first and second groups defined at step f) derive; g) reiterating steps f) and g) until every arbitrary quantification value provided at step d) is tested; h) setting the said predetermined reference value as consisting of the arbitrary quantification value for which the highest statistical significance (most significant P-value obtained with a log-rank test, significance when P<0.05) has been calculated at step g).

For example the expression of SPARC has been assessed for 100 biological samples of 100 subjects. The 100 samples are ranked according to the number of cells. Sample 1 has the highest number and sample 100 has the lowest number. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer subject, Kaplan-Meier curves are prepared for each of the 99 groups of two subsets. Also for each of the 99 groups, the p value between both subsets was calculated (log-rank test). The predetermined reference value is then selected such as the discrimination based on the criterion of the minimum P-value is the strongest. In other terms, the cell density corresponding to the boundary between both subsets for which the P-value is minimum is considered as the predetermined reference value. It should be noted that the predetermined reference value is not necessarily the median value of cell densities. Thus in some embodiments, the predetermined reference value thus allows discrimination between a poor and a good prognosis with respect to DFS and OS for a subject. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P-value) are retained, so that a range of quantification values is provided. This range of quantification values includes a “cut-off value as described above. For example, according to this specific embodiment of a “cut-off value, the outcome can be determined by comparing the cell density with the range of values which are identified. In some embodiments, a cut-off value thus consists of a range of quantification values, e.g. centered on the quantification value for which the highest statistical significance value is found (e.g. generally the minimum P-value which is found).

### Method for Treating a Subject as Defined Having Short Survival

In a second aspect, the invention relates to a method for treating a subject as defined having short survival according to the invention with an anti-SPARC-targeted therapy.

In a particular embodiment, the method according to the invention wherein the subject has a positive expression of SPARC in CAFs. Typically, such subject is defined as SPARC+CAFs.

More particularly, the invention relates to a method for treating a subject as defined having short survival comprising a step of administering to said subject a therapeutically effective amount of an inhibitor of SPARC.

In a particular embodiment, the method according to the invention wherein the subject is suffering or is susceptible to suffer from TNBC non-metastatic or metastatic. OK

As used herein, the term “subject” refers to any mammals, such as a rodent, a feline, a canine, and a primate. Particularly, in the present invention, the subject is a human. In a particular embodiment, the subject is a human who is susceptible to have TNBC. More particularly, said subject is a human having a positive expression of SPARC in CAFs (SPARC+CAFs).

In a particular embodiment, the invention relates to a method of treating TNBC in a subject in need thereof comprising i) a first step consisting in determining whether the subject has a short survival time according to methods as described above and ii) administering to said subject a therapeutically amount of inhibitor of SPARC.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is mean the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

A “therapeutically effective amount” is intended for a minimal amount of active agent which is necessary to impart therapeutic benefit to a subject. For example, a “therapeutically effective amount” to a subject is such an amount which induces, ameliorates or otherwise causes an improvement in the pathological symptoms, disease progression or physiological conditions associated with or resistance to succumbing to a disorder. It will be understood that the total daily usage of the compounds of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidential with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient (inhibitor of SPARC) for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

In some embodiments, the treatment consists of administering to the subject a targeted cancer therapy. Targeted cancer therapies are drugs or other substances that block the growth and spread of cancer by interfering with specific molecules (“molecular targets”) that are involved in the growth, progression, and spread of cancer. Targeted cancer therapies are sometimes called “molecularly targeted drugs,” “molecularly targeted therapies,” “precision medicines,” or similar names.

In a particular embodiment, the treatment consists of administering to the subject a SPARC targeted therapy.

As used herein, the term “SPARC targeted therapy” refers to drugs, other substances or inhibitors that interfere, block, or inhibit SPARC expression at nucleic or protein level.

As used herein, the term “inhibitors of SPARC” refers to a natural or synthetic compound that has a biological effect to inhibit or significantly reduce the activity or expression of the transcripts and/or proteins. Thus, an “inhibitor of SPARC” refers to a natural or synthetic compound that has a biological effect to inhibit or significantly reduce the activity or expression of SPARC transcripts and/or proteins. In a particular embodiment, the inhibitor of SPARC is an inhibitor of SPARC activity. The term “inhibitor of SPARC activity” has its general meaning in the art and refers to a compound that has the capability of reducing or suppressing selectively the activity of SPARC. Typically, an inhibitor of SPARC activity is a small organic molecule, a polypeptide, an aptamer or an antibody.

In some embodiments, the inhibitor of SPARC activity is a small organic molecule. The term “small organic molecule” as used herein, refers to a molecule of a size comparable to those organic molecules generally used in pharmaceuticals. The term excludes biological macromolecules (e. g. proteins, nucleic acids, etc.). Typically, small organic molecules range in size up to about 5000 Da, more preferably up to 2000 Da, and most preferably up to about 1000 Da.

In some embodiments, the inhibitor of SPARC activity is an antibody. More particularly, the antibody is suitable to inhibit SPARC. The term “antibody” is thus used to refer to any antibody-like molecule that has an antigen binding region, and this term includes antibody fragments that comprise an antigen binding domain such as Fab′, Fab, F(ab′)2, single domain antibodies (DABs or VHH), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa (lamda) bodies (scFv-CL fusions); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art. In some embodiments, the antibody is a monoclonal antibody. In some embodiments, the antibody is non-internalizing. As used herein the term “non-internalizing antibody” refers to an antibody, respectively, that has the property of to bind to a target antigen present on a cell surface, and that, when bound to its target antigen, does not enter the cell and become degraded in the lysosome. Particularly, in the context of the invention, the antibody is a single domain antibody. The term “single domain antibody” has its general meaning in the art and refers to the single heavy chain variable domain of antibodies of the type that can be found in Camelid mammals which are naturally devoid of light chains. Such single domain antibody are also called VHH or “Nanobody®”. For a general description of (single) domain antibodies, reference is also made to the prior art cited above, as well as to EP 0 368 684, Ward et al. (Nature 1989 Oct. 12; 341 (6242): 544-6), Holt et al, Trends BiotechnoL, 2003, 21 (11): 484-490; and WO 06/030220, WO 06/003388. In the context of the invention, the amino acid residues of the single domain antibody are numbered according to the general numbering for VH domains given by the International ImMunoGeneTics information system aminoacid numbering (htt://imgt.cines.fr/). Particularly, in the context of the invention, the antibody is a single chain variable fragment. The term “single chain variable fragment” or “scFv fragment” refers to a single folded polypeptide comprising the VH and VL domains of an antibody linked through a linker molecule. In such a scFv fragment, the VH and VL domains can be either in the VH-linker-VL or VL-linker-VH order. In addition to facilitate its production, a scFv fragment may contain a tag molecule linked to the scFv via a spacer. A scFv fragment thus comprises the VH and VL domains implicated into antigen recognizing but not the immunogenic constant domains of corresponding antibody.

In some embodiments, the inhibitor of SPARC activity is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In some embodiments, the inhibitor of SPARC activity is a polypeptide. The term “polypeptide” refers to a polypeptide that specifically bind to SPARC, can be used as a SPARC inhibitor that bind to and sequester the SPARC protein, thereby preventing it from signaling. Polypeptide refers both short peptides with a length of at least two amino acid residues and at most 10 amino acid residues, oligopeptides (11-100 amino acid residues), and longer peptides (the usual interpretation of “polypeptide”, i.e. more than 100 amino acid residues in length) as well as proteins (the functional entity comprising at least one peptide, oligopeptide, or polypeptide which may be chemically modified by being glycosylated, by being lipidated, or by comprising prosthetic groups). The definition of polypeptides also comprises native forms of peptides/proteins in mycobacteria as well as recombinant proteins or peptides in any type of expression vectors transforming any kind of host, and also chemically synthesized peptides.

In a particular embodiment, the inhibitor of SPARC is an inhibitor of SPARC expression. An “inhibitor of SPARC expression” refers to a natural or synthetic compound that has a biological effect to inhibit or significantly reduce the expression of the gene encoding for SPARC. Typically, the inhibitor of SPARC expression has a biological effect on one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

In some embodiments, the inhibitor of SPARC expression is an antisense oligonucleotide. Anti-sense oligonucleotides, including anti-sense RNA molecules and anti-sense DNA molecules, would act to directly block the translation of SPARC mRNA by binding thereto and thus preventing protein translation or increasing mRNA degradation, thus decreasing the level of SPARC proteins, and thus activity, in a cell. For example, antisense oligonucleotides of at least about 15 bases and complementary to unique regions of the mRNA transcript sequence encoding SPARC can be synthesized, e.g., by conventional phosphodiester techniques and administered by e.g., intravenous injection or infusion. Methods for using antisense techniques for specifically alleviating gene expression of genes whose sequence is known are well known in the art (e.g. see U.S. Pat. Nos. 6,566,135; 6,566,131; 6,365,354; 6,410,323; 6,107,091; 6,046,321; and 5,981,732).

In some embodiments, the inhibitor of SPARC expression is a small inhibitory RNAs (siRNAs). SPARC expression can be reduced by contacting the subject or cell with a small double stranded RNA (dsRNA), or a vector or construct causing the production of a small double stranded RNA, such that SPARC expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836).

In some embodiments, inhibitor of SPARC expression is a ribozyme. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of SPARC mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

In some embodiments, the inhibitor of SPARC expression is an endonuclease. The term “endonuclease” refers to enzymes that cleave the phosphodiester bond within a polynucleotide chain. Some, such as Deoxyribonuclease I, cut DNA relatively nonspecifically (without regard to sequence), while many, typically called restriction endonucleases or restriction enzymes, and cleave only at very specific nucleotide sequences. The mechanism behind endonuclease-based genome inactivating generally requires a first step of DNA single or double strand break, which can then trigger two distinct cellular mechanisms for DNA repair, which can be exploited for DNA inactivating: the errorprone nonhomologous end-joining (NHEJ) and the high-fidelity homo logy-directed repair (HDR). In a particular embodiment, the endonuclease is CRISPR-cas. As used herein, the term “CRISPR-cas” has its general meaning in the art and refers to clustered regularly interspaced short palindromic repeats associated which are the segments of prokaryotic DNA containing short repetitions of base sequences. In some embodiment, the endonuclease is CRISPR-cas9 which is from Streptococcus pyogenes. The CRISPR/Cas9 system has been described in U.S. Pat. No. 8,697,359 B1 and US 2014/0068797. In some embodiment, the endonuclease is CRISPR-Cpf1 which is the more recently characterized CRISPR from Provotella and Francisell.

Thus, in some embodiments, the inhibitor of SPARC fragment inhibits the migration, endothelial transmigration and invasion of cancer cells

In some embodiments, the inhibitor of SPARC block the oncogenic action of SPARC

In a further embodiment, the invention relates to a method for treating a subject suffering from TNBC compressing a step of administering to said subject: i) anti-cancer therapy and ii) a SPARC inhibitor, as a combined preparation.

In a particular embodiment, the method according to the invention wherein the i) anti-cancer therapy and ii) the SPARC inhibitor are administered simultaneously, sequentially or separately.

As used herein, the term “anti-cancer therapy” refers to treatment to stop or prevent a cancer. In the context of the invention anti-cancer therapy allows to treat and/or prevent TNBC. In a particular embodiment, the anti-cancer therapy includes but not limited to chemotherapy, radiation therapy, surgery, immunotherapy, and others.

As used herein, the terms “combined treatment”, “combined therapy” or “therapy combination” refer to a treatment that uses more than one medication. The combined therapy may be dual therapy or bi-therapy.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

As used herein, the term “chemotherapy” refers to cancer treatment that uses one or more chemotherapeutic agents. As used herein, the term “chemotherapeutic agent” refers to chemical compounds that are effective in inhibiting tumor growth. Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaorarnide and trimethylolomelamine; acetogenins (especially bullatacin and bullatacinone); a camptothecin (including the synthetic analogue topotecan and irinotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estrarnustine, ifosfamide, mechlorethamine, mechlorethamine oxide hydrochloride, melphalan, novembichin, phenesterine, prednimus tine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin (11 and calicheamicin 211, see, e.g., Agnew Chem Intl. Ed. Engl. 33:183-186 (1994); dynemicin, including dynemicin A; an esperamicin; as well as neocarzinostatin chromophore and related chromoprotein enediyne antiobiotic chromomophores), aclacinomysins, actinomycin, authramycin, azaserine, bleomycins, cactinomycin, carabicin, canninomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idanrbicin, marcellomycin, mitomycins, mycophenolic acid, nogalarnycin, olivomycins, peplomycin, potfiromycin, puromycin, quelamycin, rodorubicin, streptomgrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, trifluridine, tipiracil, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophospharnide glycoside; aminolevulinic acid; amsacrine; bestrabucil; bisantrene; edatraxate; defo famine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pento statin; phenamet; pirarubicin; podophyllinic acid; 2-ethylhydrazide; procarbazine; PSK®; razoxane; rhizoxin; sizofiran; spirogennanium; tenuazonic acid; triaziquone; 2,2′,2″-trichlorotriethylarnine; trichothecenes (especially T-2 toxin, verracurin A, roridinA and anguidine); urethan; vindesine; dacarbazine; mannomustine; mitobromtol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.].) and doxetaxel (TAXOTERE®, Rhone-Poulenc Rorer, Antony, France); chlorambucil; gemcitabine; 6-thioguanine; mercaptopurine; methotrexate; platinum analogs such as cisp latin and carbop latin; vinblastine; platinum such as oxaliplatin, cisplatin and carbloplatin; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylornithine (DMFO); retinoic acid; capecitabine; ziv-aflibercept; and pharmaceutically acceptable salts, acids or derivatives of any of the above. Also included in this definition are antihormonal agents that act to regulate or inhibit honnone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

As used herein, the term “radiation therapy” has its general meaning in the art and refers the treatment of cancer with ionizing radiation. Ionizing radiation deposits energy that injures or destroys cells in the area being treated (the target tissue) by damaging their genetic material, making it impossible for these cells to continue to grow. One type of radiation therapy commonly used involves photons, e.g. X-rays. Depending on the amount of energy they possess, the rays can be used to destroy cancer cells on the surface of or deeper in the body. The higher the energy of the x-ray beam, the deeper the x-rays can go into the target tissue. Linear accelerators and betatrons produce x-rays of increasingly greater energy. The use of machines to focus radiation (such as x-rays) on a colorectal cancer site is called external beam radiation therapy. Gamma rays are another form of photons used in radiation therapy. Gamma rays are produced spontaneously as certain elements (such as radium, uranium, and cobalt 60) release radiation as they decompose, or decay. In some embodiments, the radiation therapy is external radiation therapy. Examples of external radiation therapy include, but are not limited to, conventional external beam radiation therapy; three-dimensional conformal radiation therapy (3D-CRT), which delivers shaped beams to closely fit the shape of a tumor from different directions; intensity modulated radiation therapy (IMRT), e.g., helical tomotherapy, which shapes the radiation beams to closely fit the shape of a tumor and also alters the radiation dose according to the shape of the tumor; conformal proton beam radiation therapy; image-guided radiation therapy (IGRT), which combines scanning and radiation technologies to provide real time images of a tumor to guide the radiation treatment; intraoperative radiation therapy (IORT), which delivers radiation directly to a tumor during surgery; stereotactic radiosurgery, which delivers a large, precise radiation dose to a small tumor area in a single session; hyperfractionated radiation therapy, e.g., continuous hyperfractionated accelerated radiation therapy (CHART), in which more than one treatment (fraction) of radiation therapy are given to a subject per day; and hypofractionated radiation therapy, in which larger doses of radiation therapy per fraction is given but fewer fractions.

As used herein, the term “immunotherapy” refers to a treatment having an action on the immune system to treat cancer, such as TNBC.

In a particular embodiment, the immunotherapy treatment is selected from the group consisting of therapeutic treatments that stimulate the subject's immune system to attack the malignant tumor cells, immunization of the subject with tumoral antigens, administration of molecules stimulating the immune system such as cytokines, administration of therapeutic antibodies, adoptive T cell therapy, immune checkpoint inhibitor treatment, and any combination thereof, particularly an immune checkpoint inhibitor treatment.

An important part of the immune system is its ability to tell between normal cells in the body and those it sees as “foreign”, in particular cancer cells. This lets the immune system attack the cancer cells while leaving the normal cells alone. To do this, the immune system uses “checkpoints”, these checkpoints are molecules on certain immune cells that need to be activated (or inactivated) to start an immune response. Cancer cells sometimes find ways to use these checkpoints to avoid being attacked by the immune system. As used herein, the term “immune checkpoint inhibitor treatment” refers to an immunotherapy treatment that target these checkpoints in order to allow or facilitate the attack of cancer cells by the immune system.

Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors.

These proteins are responsible for co-stimulatory or inhibitory interactions of T-cell responses. Thus, immune checkpoint proteins regulate and maintain self-tolerance and the duration and amplitude of physiological immune responses.

Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein. Inhibition includes reduction of function and full blockade.

In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. In natural antibodies, two heavy chains are linked to each other by disulfide bonds and each heavy chain is linked to a light chain by a disulfide bond. There are two types of light chain, lambda (l) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa (lamda) bodies (scFv-CL fusions); BiTE (Bispecific T-cell Engager, scFv-scFv tandems to attract T cells); DVD-Ig (dual variable domain antibody, bispecific format); SIP (small immunoprotein, kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/1 1 161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388.

In a particular embodiment, the immune checkpoint inhibitor is a monoclonal antibody.

Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique. Typically, antibodies are directed against A2AR, B7-H3, B7-H4, BTLA, CTLA-4, CD277, IDO, KIR, PD-1, LAG-3, TIM-3 or VISTA. In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK). In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS). In some embodiments, the immune checkpoint inhibitor is an anti-PD-L2 antibody such as described in U.S. Pat. Nos. 7,709,214, 7,432,059 and 8,552,154. In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macromolecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, β-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to -N-(3-bromo-4-fluorophényl)-N′-hydroxy-4-{[2-(sulfamoylamino)-éthyl]amino}-1,2,5-oxadiazole-3 carboximidamide:

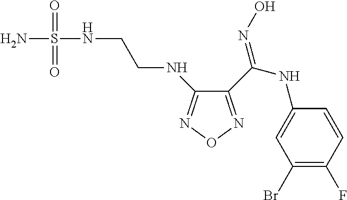 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7] cyclohepta [1,2-c]pyridazin-3-yl)-N3-[(7S)-6,7,8,9-tetrahydro-7-(1-pyrrolidinyl)-5H-benzocyclohepten-2-yl]- and has the following formula in the art:

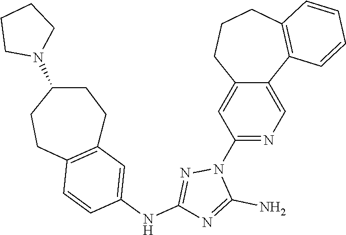 [SMILES: Nc1nc(Nc2ccc3c(c2)CC[C@@H](N2CCCC2)CC3)nn1-c1cc2c(cn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by Exponential enrichment (SELEX) of a random sequence library. The random sequence library is obtainable by combinatorial chemical synthesis of DNA. In this library, each member is a linear oligomer, eventually chemically modified, of a unique sequence.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

In a particular embodiment, the invention relates to an i) anti-PD1, and ii) anti-SPARC-targeted therapy, as a combined preparation for simultaneous, separate or sequential use in the treatment of TNBC.

The combined preparation for use according to the invention wherein anti-PD1 is selected from the following group but not limited to: Nivolumab (Opdivo®, BMS), Pembrolizumab (also called Lambrolizumab, KEYTRUDA® or MK-3475, MERCK).

In a particular embodiment, the invention relates to an i) anti-PDL1, and ii) anti-SPARC-targeted therapy, as a combined preparation for simultaneous, separate or sequential use in the treatment of TNBC.

The combined preparation for use according to the invention wherein anti-PDL1 is selected from the following group but not limited to: Atezolizumab (MPDL3280A, Genentech/Roche), Durvalumab (AZD9291, AstraZeneca), Avelumab (also known as MSB0010718C, Merck) and BMS-936559 (BMS)).

### Pharmaceutical Composition

The SPARC inhibitors and/or their combination with anti-cancer therapy as described above may be combined with pharmaceutically acceptable excipients, and optionally sustained-release matrices, such as biodegradable polymers, to form pharmaceutical compositions.

Accordingly, in a third aspect, the invention relates to a pharmaceutical composition comprising SPARC inhibitors and/or their combination with anti-cancer therapy. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and freeze-drying techniques which yield a powder of the active ingredient plus any additional desired ingredient from a previously sterile-filtered solution thereof. Upon formulation, solutions will be administred in a manner compatible with the dosage formulation and in such amount as is therapeutically effective. The formulations are easily administred in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

### Kit for Predicting and Treating TNBC

In a fourth aspect, the invention relates to a kit suitable to predict the survival time of a subject suffering or susceptible to suffer from TNBC.

Accordingly, the invention relates to a kit for use in the method for predicting the survival time of a subject having or susceptible to have TNBC said kit comprising a reagent that specifically reacts with SPARC mRNA or protein in CAFs and instructions use to perform the predicting method of the survival time according to the method as described above.

The kit for the use according to the invention, wherein the reagent that specifically reacts with SPARC mRNA or protein in CAFs is selected from the group consisting of oligonucleotide probes that specifically hybridize to SPARC transcripts, oligonucleotide primers that specifically amplify SPARC transcripts, antibodies that specifically recognize/bind the SPARC protein.

### Method of Screening

In a fifth aspect, the present invention relates to a method of screening a drug suitable for the treatment of TNBC comprising i) providing a test compound and ii) determining the ability of said test compound to inhibit the expression and/or activity of SPARC in CAFs.

Any biological assay well known in the art could be suitable for determining the ability of the test compound to inhibit the expression and/or activity of SPARC in CAFs. In some embodiments, the assay first comprises determining the ability of the test compound to bind to SPARC in CAFs. Binding to SPARC and inhibition of the biological activity of SPARC may be determined by any competing assays well known in the art. For example, the assay may consist in determining the ability of the agent to be tested as inhibitor of SPARC to bind to SPARC. The binding ability is reflected by the Kd measurement. The term “KD”, as used herein, is intended to refer to the dissociation constant, which is obtained from the ratio of Kd to Ka (i.e. Kd/Ka) and is expressed as a molar concentration (M). KD values for binding biomolecules can be determined using methods well established in the art. In specific embodiments, an inhibitor of SPARC that binds to SPARC is intended to refer to an inhibitor that binds to human SPARC with a KD of 1 μM or less, 100 nM or less, 10 nM or less, or 3 nM or less. Then a competitive assay may be settled to determine the ability of the agent to inhibit biological activity of SPARC. The functional assays may be envisaged such evaluating the ability to inhibit migration and/or endothelial transmigration and/or invasion of cancer cells.

In some embodiments, a population of cells is then contacted and activated so as to determine the ability of the test compound to inhibit the activity of SPARC. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity of SPARC in CAFs, as determined using in vitro methods described herein, are likely to exhibit similar modulatory capacity in applications in vivo. Typically, the test compound is selected from the group consisting of peptides, petptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1. SPARC is a biomarker in TNBC and its expression in CAFs predicts RFS in TNBC. (A) Quantification of SPARC expression in TNBC stroma. Percentage of TNBC samples with positive SPARC signal (>50% of stained cells) in the indicated stromal cell types. N=148 samples. (B) Quantification of SPARC expression in normal breast. Percentage of normal breast tissue samples in which SPARC expression was lower (N<T), similar (=) or higher (N>T) than in the adjacent TNBC. T, tumor; N, normal breast; n=50 samples. (C) Relapse-free survival according to SPARC expression status in CAFs. Patients with TNBC were divided in two subgroups according to SPARC expression in CAFs: SPARC+CAFs and SPARC− CAFs.

FIG. 2. Relapse-free survival in function of the SPARC expression status in TNBC cancer cells. Patients with TNBC were divided in two subgroups according to SPARC expression in tumor cells: SPARC+ and SPARC−.

FIG. 3. Relapse-free survival in function of SPARC status in TAMs. Patients with TNBC were divided in two subgroups according to SPARC expression in TAMs: SPARC⁺ and SPARC⁻.

FIG. 4. Relapse-free survival according in function of SPARC expression status in endothelial cells. Patients with TNBC were divided in two subgroups according to SPARC expression in endothelial cells within the tumor microenvironment: SPARC⁺ and SPARC⁻.

FIG. 5. Relapse-free survival according to SPARC expression status in TILs. Patients with TNBC were divided in two subgroups according to SPARC expression in TILs: SPARC⁺ and SPARC⁻.

FIG. 6. Effects of fibroblast-secreted SPARC on TNBC cell migration induced by wound healing. MDA-MB-231 sub-confluent cell layers were wounded using the 96-well IncuCyte® scratch wound assay. Wound healing (wound width, in μm) in the presence of HMF CM or SPARC-immunodepleted HMF CM was quantified over time. The data are the mean±SD (n=3): ***p<0.001 (Student's t-test). Similar results were obtained in another independent experiment.

FIG. 7. Effects of fibroblast-secreted SPARC on TNBC cell invasion. Cell invasion in tumor spheroid assay: MDA-MB-231 tumor spheroids embedded in collagen I gel were let to invade in the presence of HMF CM or SPARC-immunodepleted HMF CM for 3 days. The invading MDA-MB-231 cell area was quantified using Image J. Data are the mean±SD (n=5); **p<0.01 (Student's t-test).

### EXAMPLE

### Material & Methods

### Antibodies and Reagents

The rabbit polyclonal anti-SPARC (15274-1-AP) and the mouse monoclonal anti-periostin (clone No 1A11A3) antibodies were purchased from Proteintech. The mouse monoclonal anti-human SPARC (clone AON-5031, sc-73472) and the mouse monoclonal anti-HSC70 (clone B-6, sc-7298) antibodies were purchased from Santa Cruz Biotechnology. The mouse monoclonal anti-tubulin antibody (clone 236-10501, #A11126) was from Thermo Fisher Scientific. The horse anti-mouse immunoglobulin G (IgG)-horseradish peroxidase (#7076), and goat anti-rabbit IgG-HRP (#7074S) secondary antibodies were from Cell Signaling Technology. The donkey anti-goat HRP conjugated antibody (FT-117890) was from Interchim. The Alexa Fluor 488-conjugated anti-rabbit IgG (#Ab 150077) was purchased from Abcam, and the Alexa Fluor 594-conjugated anti-mouse IgG (711-585-152) from ImmunoResearch Laboratories. Hoechst 33342 (#FP-BB1340) was from Interchim FluoProbes.

### Patients and Tumor Samples

For TNBC cytosols, patient samples were processed according to the French Public Health Code (law n°2004-800, articles L. 1243-4 and R. 1243-61), and the biological resources center has been authorized (authorization number: AC-2008-700; Val d'Aurelle, ICM, Montpellier) to deliver human samples for scientific research. TNBC tissue micro-arrays (TMAs) included tissue samples from 148 patients with unifocal, unilateral, non-metastatic TNBC who underwent surgery at Montpellier Cancer Institute between 2002 and 2012. TNBC samples were provided by the Biological Resource Center (Biobank number BB-0033-00059) after approval by the Montpellier Cancer Institute Institutional Review Board, following the French Ethics and Legal regulations for the patients' information and consent. All patients were informed before surgery that their surgical specimens might be used for research purposes. Patients did not receive neoadjuvant chemotherapy before surgery. ER and PR negativity were defined as <10% expression by immunohistochemistry (IHC), and HER2 negativity was defined as IHC 0/1+ or 2+ and negative fluorescent/chromogenic hybridization in situ. This study was reviewed and approved by the Montpellier Cancer Institute Institutional Review Board (ID number ICM-CORT-2016-04). The study approval for patient-derived xenografts (PDXs) was previously published [38].

### Construction of TNBC TMAs

Tumor tissue blocks with enough material at gross inspection were selected from the Biological Resource Center. The presence of tumor tissue in sections was evaluated by a pathologist after hematoxylin-eosin (HE) staining of few sections. Two representative tumor areas were identified on each slide from which two malignant cores (1 mm in diameter) were extracted with a manual arraying instrument (Manual Tissue Arrayer 1, Beecher Instruments, Sun Prairie, WI, USA). After arraying completion, 4 μm sections were cut from the TMA blocks. One section was stained with HE and the others were used for IHC.

### TMA IHC

TMA sections were incubated with antibodies against SPARC (mouse monoclonal antibody; clone AON-5031, Santa Cruz Technology), cytokeratin 5/6 (mouse monoclonal, clone 6D5/16 B4, Dako), epidermal growth factor receptor (EGFR) (mouse monoclonal, clone 31G7, inVitroGen), PD-1 (mouse monoclonal, clone MRQ-22, BioSB), PD-L1 (rabbit monoclonal, clone SP142, Roche) and CD163 (mouse monoclonal, clone 10D6, BioSB) on a Autostainer Link48 platform (Dako) using the EnVision FLEX® system (Dako) for signal amplification and diaminobenzidine tetrahydrochloride as chromogen. TMA sections were analyzed independently by two trained observers both blinded to the clinicopathological characteristics and patient outcomes. In case of disagreement, sections were revised by a third observer to reach a consensus. Results from duplicate cores, when available, were averaged. Basal-like phenotype was defined by cytokeratin 5/6 and/or EGFR expression (>10% of tumor cells). SPARC signal in cancer cells was scored as negative (<1% of stained cells), or positive (≥1% of stained cells). SPARC signal in CAFs, TAMs, endothelial cells, and TILs was scored as negative (<50% of stained cells), or positive (≥50% of stained cells). SPARC signal in normal epithelial breast tissue samples (N) was compared to the paired tumor sample (T) and scored as lower (N<T), equal (=), or higher (N≥T). TIL density (peritumoral and intratumoral) was evaluated on HE-stained sections, and was scored as: 0 (no TILs), 1 (rare TILs), 2 (moderate infiltrate, fewer TILs than tumor cells), and 3 (diffuse infiltrate, more TILs than tumor cells). Fibrosis was evaluated on HE-stained sections, and was scored as: 0 (no CAF), >20%, 20%-50%, >50% of fibrosis. PD-1 expression by TILs was scored as follows: not evaluable (no TILs), 0 (no stained TIL), 1 (<10% of stained TILs), 2 (10-50% of stained TILs) and 3 (>50% of stained TILs). PD-L1 expression in tumor cells was considered positive if detected in ≥1% of cells. TAM density was scored in CD163-stained sections and compared to the TIL density: 0 (no TAM), 1 (rare TAMs), 2 (moderate infiltrate, fewer TAMs than TILs), 3 (diffuse infiltrate, more TAMs than TILs).

### Immunofluorescence Analysis

Paraffin-embedded patient-derived xenografts (PDX) tissue sections were deparaffined, rehydrated, rinsed, and saturated in PBS with 5% fetal calf serum (FCS) at 4° C. overnight. Sections were incubated with 1.2 μg/mL anti-SPARC rabbit polyclonal antibody (15274-1-AP) and 5 □g/mL anti-periostin mouse monoclonal antibody (1A11A3), followed by incubation with AlexaFluor 488-conjugated anti-rabbit IgG and AlexaFluor 594-conjugated anti-mouse IgG (1/400), respectively. Nuclei were stained with 0.5 μg/mL Hoechst 33342. Sections were imaged with a 63 X Plan-Apochromat objective on z stacks with a Zeiss Axio Imager light microscope equipped with Apotome to eliminate out-of-focus fluorescence.

### TNBC Cytosols, Cell Lines, Conditioned Medium, and Western Blotting

TNBC cytosols were previously prepared and frozen [39]. The MDA-MB-453, MDA-MB-436, MDA-MB-468, Hs578T, BT-549 and HCC1806 TNBC cell lines were obtained from SIRIC Montpellier Cancer. The SUM159 TNBC cell line was from Asterand (Bioscience, UK). The MDA-MB-231 TNBC cell line was previously described [40]. Human mammary fibroblasts (HMFs) were provided by J. Loncarek and J. Piette (CRCL Val d'Aurelle-Paul Lamarque, Montpellier, France) [41], THP1 monocytes by L. Gros (IRCM, Montpellier), and human umbilical vein endothelial cells (HUVECs) by M. Villalba (IRMB, Montpellier). Cell lines were cultured in DMEM with 10% FCS (EuroBio), except the SUM159 cell line (RPMI with 10% FCS) and the THP 1 cell line (RPMI with 10% decomplemented FCS, 10 mM HEPES, 1 mM sodium pyruvate and 50 □M □-mercaptoethanol). THP1 monocytes were differentiated into MO macrophages by exposure to phorbol 12-myristate 13-acetate (100 ng/ml; Sigma Aldrich) for 48h. Then, cells became adherent and the medium was replaced with fresh medium supplemented with interleukin-4 (20 ng/ml) for 24h to induce differentiation of MO macrophages to M2-polarized macrophages. For western blotting experiments, cell lysates were prepared in lysis buffer (50 mM HEPES [pH 7.5], 150 mM NaCl, 10% glycerol, 1% Triton X-100, 1.5 mM MgCl2, 1 mM EGTA) containing cOmplete™ Protease Inhibitor Cocktail (Roche, Switzerland), and centrifuged at 13,000×g for 10 min. The corresponding conditioned media were centrifuged at 500×g for 5 min. Proteins from whole cytosols (20 μg) or cell lysates (30 μg) and conditioned media (40 μl) were separated on 13.5% SDS-PAGE and analyzed by immunoblotting with the anti-SPARC (clone AON-5031) and anti-tubulin antibodies using standard techniques. To prepare conditioned medium, HMFs were grown to 90% confluence in DMEM complemented with 10% FCS. Following washes with phenol red- and serum-free medium to remove serum proteins, cells were incubated in DMEM buffered with 50 mM HEPES [pH 7.5] and without FCS for 24h. Medium was harvested, and centrifuged at 1000 rpm for 5 min, followed or not by SPARC depletion. Briefly, HMF conditioned medium was incubated with 5 μg of monoclonal anti-human SPARC antibody (clone AON-5031, sc-73472) overnight, and pre-absorbed to protein G-agarose at 4° C. Then conditioned medium (SPARC-immunodepleted or not) was filtered using 0.22 μm filters to eliminate cell debris. Cleared HMF conditioned medium (HFM CM) was collected and added to MDA-MB-231 cells for in vitro functional assays. SPARC immunodepletion was confirmed by western blotting.

### ScRNA-Seq Data Meta-Analysis

Previously published scRNA-seq data from five patients with TNBC were used [8]. Processed 10X Genomics (Pleasanton, CA, USA) data, obtained from the European Nucleotide Archive under the accession code PRJEB35405, were loaded in R (4.0) and processed using the Seurat 3.4 package and default parameters [42]. Individual cell populations were annotated as published in the original scRNA-seq study [8] with minor modifications when appropriate.

### Cell Adhesion, Migration and Invasion Assays

MDA-MB-231 cell adhesion was assessed as previously described [37]. Briefly, 96-well plates were coated with fibronectin (10 μg/ml; sc-29011; Santa Cruz Biotechnology) at 4° C. overnight, and saturated with 1% bovine serum albumin (BSA) in PBS. MDA-MB-231 cells were detached with HyQTase (HyClone), washed in DMEM without FCS, and 1.0 105 cells were pre-incubated or not with what at room temperature for 10 min. Cells (5 104 cells) were then plated and incubated in serum-free HMF CM (SPARC-immunodepleted or not) at 37° C. for 30 min. Non-adherent cells were removed by flotation on a dense Percoll solution containing 3.33% NaCl (1.10 g/l), and adherent cells were fixed (10% [vol/vol] glutaraldehyde) using the buoyancy method [43]. Cells were stained with 0.1% crystal violet, and absorbance was measured at 570 nm. For migration and invasion assays, 8-μm pore Transwell inserts (polyvinyl pyrrolidone-free polycarbonate filters) in 24-well plates (Corning Inc., Corning, NY, USA) were coated with 10 μg/ml fibronectin (500 ng) (migration assays) or Matrigel (100 μg, Corning) (invasion assays) at 4° C. for 24h. MDA-MB-231 cells were plated (5 104 cells/well) in serum-free HMF CM (SPARC-immunodepleted or not) on the coated insert in the upper chamber. In these different assays, DMEM supplemented with 10% FCS was used as chemoattractant in the bottom chamber. After 16h, non-migrating/non-invading cells on the apical side of each insert were scraped off with a cotton swab, and migration and invasion were analyzed with two methods: (1) migrating/invading cells were fixed in methanol, stained with 0.1% crystal violet for 30 min, rinsed in water, and imaged with an optical microscope. Two images of the pre-set field per insert were captured (×100); (2) migrating/invading cells were incubated with 3-(4,5-dimethylthiazol-2-yl)-2,5-diphenyltetrazolium bromide (MTT; 5 mg/ml, 1/10 volume; Sigma-Aldrich) added to the culture medium at 37° C. for 4 h. Then, the culture medium/MTT solution was removed and centrifuged at 10,000 rpm for 5 min. After centrifugation, cell pellets were suspended in DMSO. Concomitantly, 300 μl of DMSO was added to each well and thoroughly mixed for 5 min. The optical density values of stained cells (cell pellet and corresponding well) were measured using a microplate reader at 570 nm.

### Wound Healing Assay by Live Cell Imaging

Before each experiment, MDA-MB-231 cells were grown to confluence in 96-well plates in a standard CO2 incubator. The 96-pin IncuCyte® WoundMaker was used to simultaneously create precise and reproducible wounds by gently removing cells from the confluent monolayer. After washing, serum-free HMF CM (SPARC-immunodepleted or not) was added, plates were placed in the IncuCyte device and cell monolayers were scanned every hour. Wound width, wound confluence, and relative wound density were calculated using user-informed algorithms that are part of the IncuCyte™ software package. These algorithms identify the wound region and provide visual representations of the segmentation parameters.

### Tumor Spheroids

To generate tumor spheroids, 5×103 MDA-MB-231 cells/well were seeded in 150 μl complete medium in ultra-low attachment 96-well plates (Corning® 96-well Clear Round Bottom Ultra-Low Attachment Microplate, NY, USA). Plates were centrifuged at 1000 rpm for 10 min, and 3 days later each spheroid was embedded in collagen gel that included 1×DMEM, penicillin and streptomycin, 2% of SPARC-immunodepleted FCS, 3.75 g/l sodium bicarbonate, 20 mM Hepes, 1 mg/ml rat collagen I, and 1.5 mM NaOH (qsp 150 □l/well in H2O). After 30 min at 37° C., serum-free HMF CM (SPARC-immunodepleted or not) was added on the spheroid-containing polymerized collagen gel. MDA-MB-231 cell invasion area was analyzed in representative images with ImageJ.

### Statistical Analyses

Data were described using means, medians and ranges for continuous variables, and frequencies and percentages for categorical variables. Data were compared with the Kruskal-Wallis test (continuous variables) and the chi-square or Fisher's exact test, if appropriate (categorical variables). All tests were two-sided, and p-values <0.05 were considered significant. The median follow-up was calculated using the reverse Kaplan-Meier method. Relapse-free survival (RFS) and OS were estimated using the Kaplan-Meier method and compared with the log-rank test. RFS was defined as the time between the date of the first histology analysis and the date of the first recurrence at any site. Surviving patients without recurrence and patients lost to follow-up were censored at the time of the last follow-up or last documented visit. OS was defined as the time between the date of the first histology analysis and the date of death from any cause. Multivariate analyses were performed using the Cox proportional hazard model. Hazard ratios (HR) were given with their 95% confidence interval (CI). All statistical analyses were performed with the STATA 13.0 software (StatCorp, College Station, TX).

### Results

### In TNBC, SPARC is Expressed in Stromal and Tumor Cells

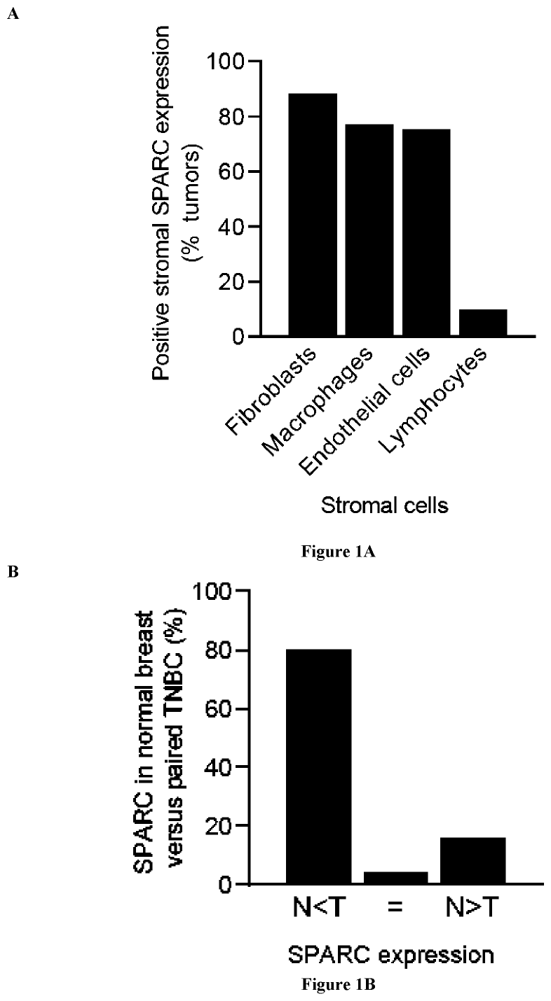

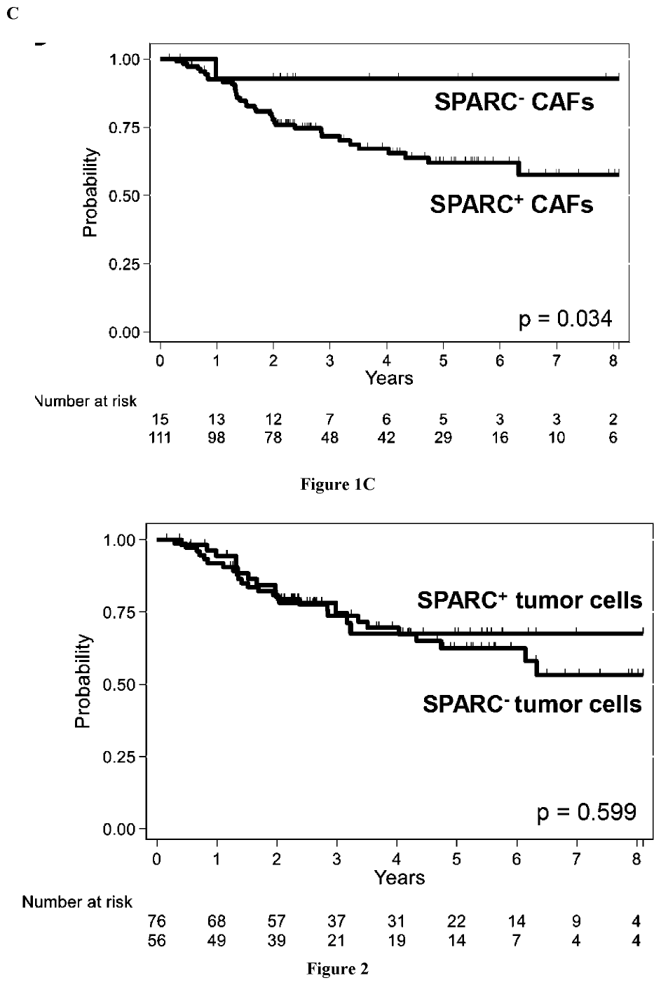

To determine SPARC expression in TNBC (tumor and stroma), TMAs were generated using samples from 148 patients with TNBC (Table 1). Their median age was 61.5 years (range 30.2-98.6), and 68.2% of them received adjuvant chemotherapy. Most TNBC (52.7%) were pT2, and 60.8% pN0. Moreover, 85.5% of tumors were ductal carcinomas, 6.9% lobular carcinomas, and 7.6% other histological types; 11% of tumors were classified as Scarff-Bloom-Richardson grade 1-2. A basal-like phenotype was observed in 61.9% of samples, and 66.9% of tumors expressed PD-L1. In 51.7% of tumors, TAMs were more abundant than TILs, and >20% of fibrosis was observed in 74.4% of tumors. SPARC expression (>50% of stained cells) in CAFs, TAMs, endothelial cells, and TILs was detected in 88.1%, 77.1%, 75.2%, and 9.8% of TNBC samples, respectively (FIG. 1A, Table 1). SPARC staining in tumor cells (>1% stained tumor cells) was observed in 42.4% of TNBC samples (Table 1). In 80% of samples, SPARC expression was lower in the adjacent normal breast tissue than in the tumor tissue (FIG. 1B).

TABLE 1
Clinicopathological characteristics of the whole population

| | Overall | | | Overall | |
|---|---|---|---|---|---|
| | population | | | population | |
| Clinical and tumors characteristics | N = 148 | | Clinical and tumors characteristics | N = 148 | |
| Age (years), median [min-max] | | | SPARC expression TAMs | | |
| <55 years | 61.5 | [30.2-98.6] | Negative | 27 | (22.9%) |
| ≥55 years | 51 | (34.5%) | Positive | 91 | (77.1%) |
| | 97 | (65.5%) | Missing | 30 | |
| Tumor size | | | SPARC expression endothelial cells | | |
| T1 | 52 | (35.1%) | Negative | 27 | (24.8%) |
| T2 | 78 | (52.7%) | Positive | 82 | (75.2%) |
| T3/T4 | 18 | (12.2%) | Missing | 39 | |
| Nodal status | | | SPARC expression TILs | | |
| N− | 90 | (60.8%) | Negative | 74 | (90.2%) |
| N+ | 58 | (39.2%) | Positive | 8 | (9.8%) |
| | | | Missing | 66 | |
| Histological grade (SBR) | | | TILs density | | |
| 1-2 | 16 | (11.0%) | [0-1] | 42 | (29.6%) |
| 3 | 130 | (89.0%) | >1 | 100 | (70.4%) |
| Missing | 2 | | Missing | 6 | |
| Histology | | | PDL-1 expression tumor cells | | |
| Ductal | 124 | (85.5%) | <1% | 45 | (33.1%) |
| Lobular | 10 | (6.9%) | ≥1% | 91 | (66.9%) |
| Other | 11 | (7.6%) | Missing | 12 | |
| Missing | 3 | | | | |
| Adjuvant chemotherapy | | | PDL-1 expression TILs | | |
| No | 47 | (31.8%) | 0 | 20 | (14.9%) |
| Yes | 101 | (68.2%) | 10-10[ | 32 | (23.9%) |
| | | | [10-50[ | 40 | (29.9%) |
| | | | ≥50 | 42 | (31.3%) |
| | | | Missing | 14 | |
| Basal-like phenotype | | | PD1 expression TIL's | | |
| ≤10% | 56 | (38.1%) | 0 | 18 | (12.9%) |
| Basal | 91 | (61.9%) | <10 | 30 | (21.3%) |
| Missing | | | [10,50[ | 74 | (52.9%) |
| | | | ≥50 | 18 | (12.9%) |
| | | | Missing | 8 | |
| SPARC expression tumor cells | | | Fibrosis | 4 | (3.0%) |
| Negative | 76 | (57.6%) | 0 | 31 | (22.6%) |
| Positive | 56 | (42.4%) | <20% | 27 | (19.7%) |
| Missing | 16 | | 20%-50% | 75 | (54.7%) |
| | | | >50% | 11 | |
| | | | Missing | | |
| SPARC expression CAFs | | | TAMs (inflammation) | | |
| Negative | 15 | (11.9%) | 0/1 | 25 | (17.5%) |
| Positive | 111 | (88.1%) | 2 | 44 | (30.8%) |

| | | | | |
|---|---|---|---|---|
| Missing | 22 | 3 | 74 | (51.7%) |

| | |
|---|---|
| Missing | 5 |

SBR: Scarff-Bloom-Richardson;
CAFs: cancer-associated fibroblasts;
TAMs: tumor-associated macrophages;
TILs: tumor-infiltrating lymphocytes

TABLE 2
Univariate and multivariate logistic regression analyses of prognostic
factors for recurrence-free survival (RFS) in TNBC TMA

| | Univariate Analysis | | Multivariate Analysis | |
|---|---|---|---|---|
| Clinical and tumor characteristics | HR 95% CI | | HR 95% CI | |
| Age | N = 148 | | | |
| <55 years | 1 | | | |
| ≥55 years | 1.52 | [0.77-3.03] | | |
| | P = 0.214 | | | |
| Tumor size | N = 148 | | | |
| T1 | 1 | | | |
| T2 | 1.67 | [0.74-3.75] | | |
| T3/T4 | 5.08 | [2.07-12.47] | | |
| | P = 0.002 | | | |
| Nodal status | N = 148 | | | |
| N− | 1 | | 1 | |
| N+ | 2.77 | [1.49-5.14] | 2.96 | [1.48-5.94] |
| | P = 0.001 | | P = 0.001 | |
| Histological grade (SBR) | N = 146 | | | |
| 1-2 | 1 | | | |
| | 0.82 | [0.36-1.85] | | |
| | P = 0.645 | | | |
| Histology | N = 145 | | | |
| Ductal | 1 | | | |
| Lobular | 1.51 | [0.59-3.86] | | |
| Other | 0.77 | [0.19-3.21] | | |
| | P = 0.651 | | | |
| Adjuvant chemotherapy | N = 148 | | | |
| No | 1 | | 1 | |
| Yes | 0.43 | [0.24-0.78] | 0.35 | [0.18-0.68] |
| | P = 0.007 | | P = 0.002 | |
| Basal-like phenotype | N = 147 | | | |
| Yes | 1 | | | |
| No | 1.55 | [0.85-2.83] | | |
| | P = 0.152 | | | |
| SPARC expression tumor cells | N = 132 | | | |
| Negative | 1 | | | |
| Positive | 0.84 | [0.44-1.62] | | |
| | P = 0.599 | | | |
| SPARC expression CAFs | N = 126 | | | |
| Negative | 1 | | 1 | |
| Positive | 5.09 | [0.70-37.18] | 6.17 | [0.84-45.2] |
| | P = 0.034 | | P = 0.015 | |
| SPARC expression TAMs | N = 118 | | | |
| Negative | 1 | | | |
| Positive | 0.52 | [0.25-1.07] | | |
| | P = 0.088 | | | |
| SPARC expression endothelial cells | N = 109 | | | |
| Negative | 1 | | | |
| Positive | 0.59 | [0.29-1.21] | | |
| | P = 0.165 | | | |
| SPARC expression TILs | N = 82 | | | |
| Negative | 1 | | | |
| Positive | 0.81 | [0.19-3.46] | | |
| | P = 0.769 | | | |
| TILs density | N = 142 | | | |
| [0-1] | 1 | | | |
| >1 | 0.92 | [0.48-1.77] | | |
| | P = 0.807 | | | |
| PDL-1 expression tumor cells | N = 136 | | | |
| <1% | 1 | | | |
| ≥1% | 0.74 | [0.39-1.40] | | |
| | P = 0.360 | | | |
| PDL-1 expression TILs | N = 134 | | | |
| 0 | 1 | | | |
| 10-50[ | 2.20 | [0.66-7.40] | | |
| ≥50 | 2.12 | [0.60-7.52] | | |
| | P = 0.356 | | | |
| PD1 expression in TILs | N = 140 | | | |
| 0 | 1 | | | |
| 10-50[ | 1.28 | [0.46-3.64] | | |
| ≥50 | 0.80 | [0.20-3.21] | | |
| | P = 0.593 | | | |
| Fibrosis | N = 137 | | | |
| ≤50% | 1 | | | |
| >50% | 0.98 | [0.52-1.83] | | |
| | P = 0.948 | | | |
| TAMs (inflammation) | N = 143 | | | |
| 0/1 | 1 | | | |
| 2 | 1.97 | [0.78-4.96] | | |
| 3 | 1.14 | [0.46-2.86] | | |

P = 0.180

SBR: Scarff-Bloom-Richardson;
CAFs: cancer-associated fibroblasts;
TAMs: tumor-associated macrophages;
TILs: tumor-infiltrating lymphocytes;
HR = hazard ratio;
CI = confidence interval;
p Value in bold, statistically significant

SPARC Expression in CAFs Predicts RFS in Patients with TNBC

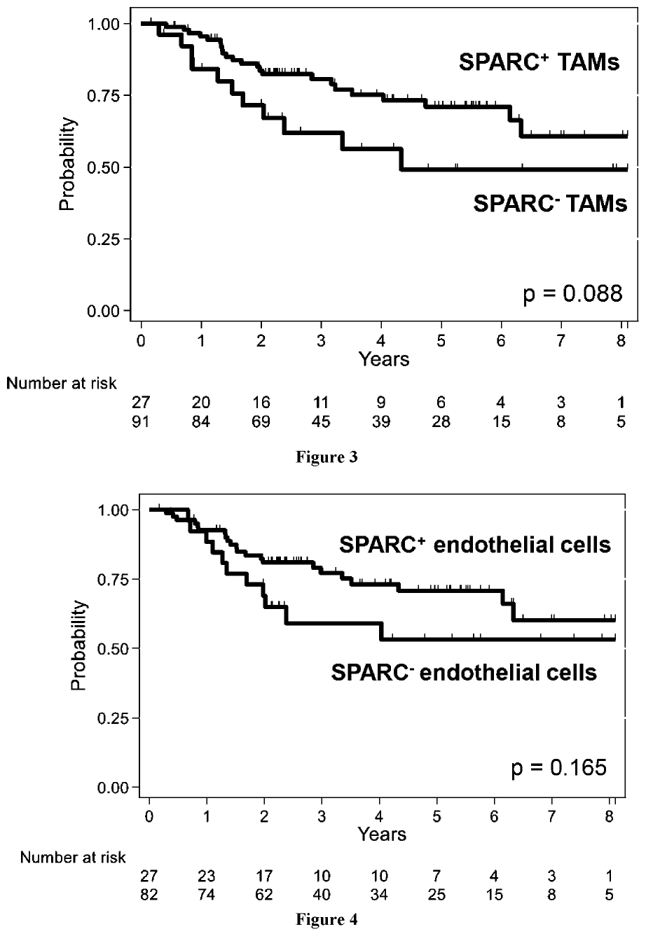

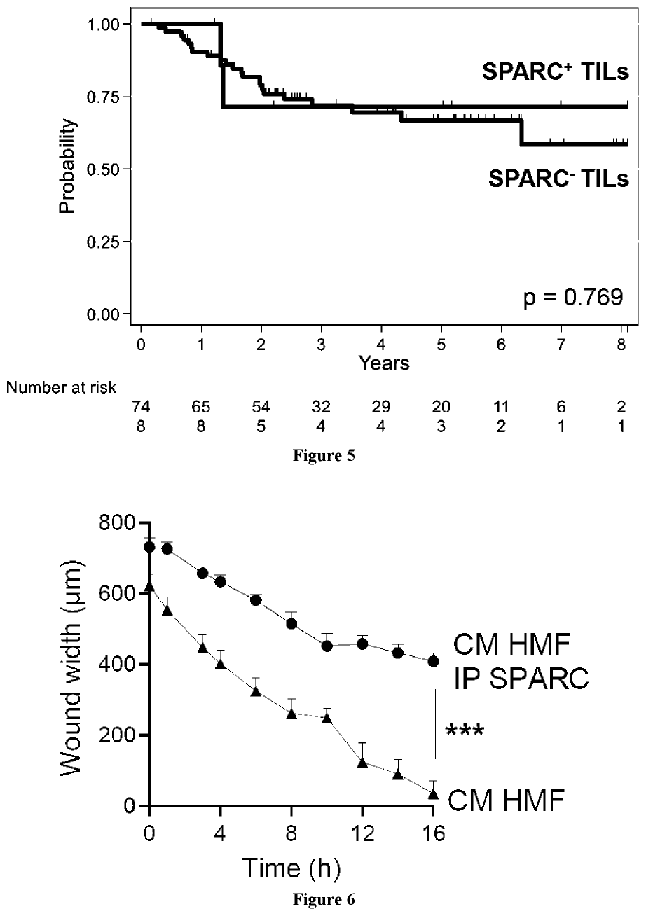

As SPARC was expressed in the tumor and stromal compartments, its prognostic value was then evaluated. The median follow-up time was 5.4 years (range [0.1-14.3]). Local or regional recurrence occurred in 10 (7%) patients, and metastases (alone or with loco-regional recurrence) in 32 (22.5%) patients. RFS was not different in patients with SPARC-positive (SPARC+) and SPARC-negative (SPARC−) tumor cells (FIG. 2) (Table 2). Conversely, RFS was lower in patients with SPARC+ than SPARC− CAFs (3-year RFS rate: 72%, 95% CI [61.5-79.6] vs. 93% (95% CI [59.1-99.0]; p=0.034) (FIG. 1C, Table 2). Moreover, RFS tended to be better in patients with SPARC+ than SPARC-TAMs (3-year RFS rate: 81%, 95% CI [70.2-87.7]) vs. 62%, 95% CI [39.2-78.2]; p=0.088) (FIG. 3). SPARC expression status in endothelial cells (FIG. 4) and TILs (FIG. 5) did not have any prognostic value (Table 2). In univariate analysis, tumor size, nodal status, adjuvant chemotherapy, and SPARC expression in CAFs were correlated with RFS (Table 2). In multivariate analysis, only nodal status (HR=2.96, 95% CI [1.48-5.94], p=0.001), adjuvant chemotherapy (HR=0.35, 95% CI [0.18-0.68], p=0.002) and SPARC expression in CAFs (HR=6.17, 95% CI [0.84-45.2], p=0.015) were independent prognostic factors of RFS (Table 2). During the follow-up, 46 (31.1%) patients died among whom 11 (7.4%) without any TNBC recurrence. In univariate analysis, age (p=0.027), tumor size (p<0.001), nodal status (p=0.002), and adjuvant chemotherapy (p=0.006) were associated with OS (data not shown). In multivariate analysis, only tumor size (p=0.05), nodal status (p=0.008), and adjuvant chemotherapy (p<0.001) were independent prognostic factors of OS (data not shown). Patients with SPARC+ CAFs (n=111, 88.1%) were younger (61.3% vs. 93.3%; p=0.018) and tended to have ductal tumors (88.0% vs. 73.3%; p=0.08) compared with patients with SPARC− CAFs (Table 3). In addition, SPARC+ TAMs and SPARC+ endothelial cells were detected more frequently in patients with SPARC+ than SPARC− CAFs (80.6% vs. 41.7%, p=0.007, and 78.0% vs. 50%, p=0.026, respectively) (Table 3). Fibrosis (>50%) was significantly less frequent in patients SPARC+ than SPARC− CAFs (48.6% vs. 80%; p=0.028) (Table 3). PD-L1 expression (>50%) in TILs was more frequently detected in patients with SPARC+ than SPARC− CAFs (34.8% vs. 15.4%; p=0.049) (Table 3). TIL density, PD-L1 expression in tumor cells and PD-1 expression in TILs were not significantly different between patients with SPARC+ and SPARC− CAFs (Table 3).

TABLE 3
Clinicopathological characteristics in function of SPARC
expression (SPARC+ and SPARC−) in CAFs

| Clinical and tumor | SPARC expression in CAFs | | SPARC expression in CAFs | | |
|---|---|---|---|---|---|
| characteristics | Negative (N = 15] | | Positive (N = 111) | | P value |
| Age | | | | | 0.018 |
| <55 years | 1 | (6.7%) | 43 | (38.7%) | |
| ≥55 years | 14 | (93.3%) | 68 | (61.3%) | |
| Tumor size | | | | | 0.334 |
| T1 | 6 | (40.0%) | 36 | (32.4%) | |
| T2 | 9 | (60.0%) | 59 | (53.2%) | |
| T3/T4 | 0 | | 16 | (14.4%) | |
| Nodal status | | | | | 0.863 |
| N- | 9 | (60.0%) | 64 | (57.7%) | |
| N+ | 6 | (40.0%) | 47 | (42.3%) | |
| Histological grade (SBR) | | | | | 0.130 |
| 1-2 | 3 | (20.0%) | 8 | (7.3%) | |
| 3 | 12 | (80.0%) | 101 | (92.7%) | |
| Histology | | | | | 0.080 |
| Ductal | 11 | (73.3%) | 95 | (88.0%) | |
| Lobular | 3 | (20.0%) | 5 | (4.6%) | |
| Other | 1 | (6.7%) | 8 | (7.4%) | |
| Adjuvant chemotherapy | | | | | 0.186 |
| No | 7 | (46.7%) | 33 | (29.7%) | |
| Yes | 8 | (53.3%) | 78 | (70.3%) | |
| ≤10% | 9 | (60.0%) | 72 | (65.4%) | |
| Basal | 6 | (40.0%) | 38 | (34.6%) | |
| SPARC expression in tumor cells | | | | | 0.603 |
| Negative | 8 | (53.3%) | 67 | (60.4%) | |
| Positive | 7 | (46.7%) | 44 | (39.6%) | |
| SPARC expression in TAMs | | | | | 0.007 |
| Negative | 7 | (58.3%) | 20 | (19.4%) | |
| Positive | 5 | (41.7%) | 83 | (80.6%) | |
| SPARC expression in endothelial | | | | | 0.026 |
| cells | | | | | |
| Negative | 7 | (50.0%) | 20 | (22.0%) | |
| Positive | 7 | (50.0%) | 71 | (78.0%) | |
| SPARC expression in TILs | | | | | 1.000 |
| Negative | 8 | (100.0%) | 65 | (89.0%) | |
| Positive | Q | | 8 | (11.0%) | |
| TIL density | | | | | 0.127 |
| [0-1] | 6 | (42.9%) | 26 | (23.9%) | |
| >1 | 8 | (57.1%) | 83 | (76.1%) | |
| PDL-1 expression in tumor cells | | | | | 0.109 |
| <1% | 7 | (53.9%) | 31 | (28.4%) | |
| ≥1% | 6 | (46.1%) | 78 | (71.6%) | |
| PDL-1 expression in TILs | | | | | 0.049 |
| 0 | 4 | (30.8%) | 10 | (9.2%) | |
| 10-50[ | 7 | (53.8%) | 61 | (56.0%) | |
| ≥50 | 2 | (15.4%) | 38 | (34.8%) | |
| PD1 expression in TILs | | | | | 0.415 |
| 0 | 2 | (15.4%) | 11 | (10.2%) | |
| 10-50[ | 8 | (61.5%) | 83 | (76.9%) | |
| 2 50 | 3 | (23.1%) | 14 | (12.9%) | |
| Fibrosis | | | | | 0.028 |
| ≤50% | 3 | (20.0%) | 56 | (51.4%) | |
| > 50% | 12 | (80.0%) | 53 | (48.6%) | |
| TAMs (inflammation) | | | | | 0.349 |
| 0/1 | 4 | (28.6%) | 15 | (13.8%) | |
| 2 | 3 | (21.4%) | 32 | (29.4%) | |
| 3 | 7 | (50.0%) | 62 | (56.9%) | |

SBR: Scarff-Bloom-Richardson;
CAFs: cancer-associated fibroblasts;
TAMs: tumor-associated macrophages;
TILs: tumor-infiltrating lymphocytes.

### SPARC Expression in TNBC Cytosols, PDX, and Cell Lines

To further validate SPARC expression in TNBC, its expression was assessed in the cytosols of 30 primary TNBC samples by western blot analysis. SPARC protein was detected in all cytosols and SPARC cleaved fragments in about 30% of samples (data not shown). SPARC protein expression and localization were then examined in two TNBC PDXs (PDX B1995 and PDX B3977) [38]. SPARC was localized in stromal cells, including CAFs, in the extracellular matrix, and in some tumor cells (data not shown). Next, SPARC expression and secretion were analyzed in TNBC and stromal cell lines. SPARC was expressed and secreted by three of the eight TNBC cell lines tested (SUM159, Hs578T, BT-549) that exhibit a basal-like phenotype (data not shown). SPARC was also expressed and secreted by HMFs, and to a lesser extent by HUVECs and M2-polarized THP1 macrophages (data not shown)).

### SPARC is Expressed in Different CAF Subsets

Based on the finding that SPARC expression in CAFs predicts RFS in TNBC, SPARC expression in different CAF subpopulations was thoroughly investigated through meta-analysis of recently published scRNA-seq data from patients with TNBC [8, 9]. In the first dataset (n=5 patients with TNBC) [8], the t-distributed Stochastic neighbor embedding (tSNE) technique identified twenty different cell populations, including two fibroblastic cell populations, the first with features of myofibroblasts (myCAFs), and the second with an inflammatory phenotype (iCAFs) characterized by high expression of growth factors and immunomodulatory molecules (data not shown). The scRNA-seq data analysis [8] showed that SPARC mRNA was strongly expressed in myCAFs and iCAFs, as well as POSTN (the gene encoding periostin, a CAF-secreted protein that promotes cancer progression and chemoresistance) (data not shown). SPARC was also detected in perivascular endothelial cells, myoepithelial cells, and basal cancer cells [8] (data not shown), in accordance with our TMA analysis (Table 1). In the second scRNA-seq dataset (n=6 patients with TNBC) [9], high SPARC and POSTN mRNA levels were detected in three distinct CAF subtypes, in endothelial cells, M2-polarized macrophages, and cancer cells (where expression varied in function of the patient) (data not shown), consistent with our TMA data (Table 1). As these two meta-analysis indicated that SPARC was expressed in different CAF subtypes, another scRNA-seq dataset (n=6 patients with breast cancer) that identified different myCAF and iCAF clusters was analyzed [10]. SPARC and POSTN mRNAs were detected mainly in myCAFs (ECM-myCAF, TGFβ-myCAF, Wound-myCAF, IFNαβ-myCAF, Acto-myCAF clusters), and also in iCAFs (IFNγ-iCAF, IL-iCAF, detox-iCAF clusters) (data not shown). Altogether, these meta-analysis highlighted that SPARC mRNA is expressed by different CAF subtypes, including myofibroblasts and inflammatory-like CAFs involved in different tumor-related processes, such as matrix remodeling, inflammation, and resistance to therapy in TNBC [8, 10]. To complement the scRNA-seq findings, the localization of SPARC and periostin was investigated in the TNBC PDX B1995 microenvironment. Co-labeling with anti-SPARC and anti-periostin antibodies showed that SPARC (in red) partially co-localized with periostin (in green) in CAFs at the cancer cell-stromal interface (data not shown).

### Fibroblast-Secreted SPARC Affects TNBC Cell Adhesion, Migration and Invasion

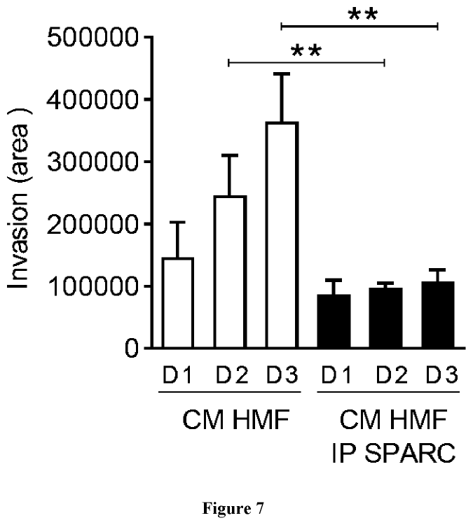

To obtain some insights into the pathophysiological relevance of SPARC+ CAFs in TNBC, the effects on TNBC cell adhesion, motility, wound healing, and invasiveness of SPARC-secreting HMF CM were investigated. The adhesion of MDA-MB-231 cells on fibronectin was reduced by 1.3-fold (p<0.001) after incubation with HMF CM compared with SPARC-immunodepleted HMF CM (data not shown). Cell motility analysis in Boyden chambers showed that 88% of MDA-MB-231 cells passed through the fibronectin-coated filters after incubation with HMF CM (data not shown). Motility was reduced by 2.3-fold when cells were incubated with SPARC-immunodepleted CM (data not shown). Moreover, wound healing was significantly faster in MDA-MB-231 cells incubated with HMF CM than with SPARC-immunodepleted CM: wound closure was nearly complete after 16h in the presence of HMF CM (FIG. 6). Lastly, MDA-MB-231 cell invasion through Matrigel-coated filters in Boyden chambers was 1.6-fold higher in the presence of HMF CM than SPARC-immunodepleted CM (data not shown). The capacity of HMF-secreted SPARC to enhance MDA-MB-231 cell invasion was confirmed in a tumor spheroid assay (FIG. 7). MDA-MB-231 tumor spheroid invasiveness at day 3 was 3.4-fold higher in the presence of HMF CM than SPARC-immunodepleted CM (FIG. 7; p<0.01). Thus, HMF-secreted SPARC inhibits adhesion and promotes motility, wound healing and invasion of MDA-MB-231 TNBC cells, highlighting its pro-tumor role.

Here inventors show that SPARC is a tumor and stromal cell-associated biomarker whose expression in CAFs independently predicts RFS in TNBC. They also show that SPARC was mainly expressed by stromal cells, including CAFs, and that its positive expression in CAFs was an independent prognostic factor of poor RFS in TNBC. Patients with SPARC⁺ CAFs had significantly more frequent PD-L1 expression on TILs, suggesting the interest to specifically evaluating the benefit of anti-PD1 or PD-L1 targeted therapies in association with anti-SPARC targeted therapy in this subgroup of TNBC patients.

### REFERENCES

1. Bianchini, G., et al., Triple-negative breast cancer: challenges and opportunities of a heterogeneous disease. Nat Rev Clin Oncol, 2016. 13 (11): p. 674-690.
2. Zheng, J. and P. Gao, Toward Normalization of the Tumor Microenvironment for Cancer Therapy. Integr Cancer Ther, 2019. 18: p. 1534735419862352.
3. Sage, E. H. and P. Bornstein, Extracellular proteins that modulate cell-matrix interactions. SPARC, tenascin, and thrombospondin. J Biol Chem, 1991. 266 (23): p. 14831-4.
4. Lane, T. F., et al., SPARC is a source of copper-binding peptides that stimulate angiogenesis. J Cell Biol, 1994. 125 (4): p. 929-43.
5 Brekken, R. A. and E. H. Sage, SPARC, a matricellular protein: at the crossroads of cell-matrix. Matrix Biol, 2000. 19 (7): p. 569-80.
6. Murphy-Ullrich, J. E. and E. H. Sage, Revisiting the matricellular concept. Matrix Biol, 2014. 37: p. 1-14.
7. Tichet, M., et al., Tumour-derived SPARC drives vascular permeability and extravasation through endothelial VCAM1 signalling to promote metastasis. Nat Commun, 2015. 6: p. 6993.
8. Hsiao, Y. H., et al., SPARC (osteonectin) in breast tumors of different histologic types and its role in the outcome of invasive ductal carcinoma. Breast J, 2010. 16 (3): p. 305-8
9. Barth, P. J., R. Moll, and A. Ramaswamy, Stromal remodeling and SPARC (secreted protein acid rich in cysteine) expression in invasive ductal carcinomas of the breast. Virchows Arch, 2005. 446 (5): p. 532-6.
10. Podhajcer, O. L., et al., The role of the matricellular protein SPARC in the dynamic interaction between the tumor and the host. Cancer Metastasis Rev, 2008. 27 (4): p. 691-705.
11. Nagaraju, G. P., et al., Molecular mechanisms underlying the divergent roles of SPARC in human carcinogenesis. Carcinogenesis, 2014. 35 (5): p. 967-73.
12. Briggs, J., et al., Transcriptional upregulation of SPARC, in response to c-Jun overexpression, contributes to increased motility and invasion of MCF7 breast cancer cells. Oncogene, 2002. 21 (46): p. 7077-91.
13. Watkins, G., et al., Increased levels of SPARC (osteonectin) in human breast cancer tissues and its association with clinical outcomes. Prostaglandins Leukot Essent Fatty Acids, 2005. 72 (4): p. 267-72.
14. Sangaletti, S., et al., Mesenchymal Transition of High-Grade Breast Carcinomas Depends on Extracellular Matrix Control of Myeloid Suppressor Cell Activity. Cell Rep, 2016. 17 (1): p. 233-248.
15. Zhu, A., et al., SPARC overexpression in primary tumors correlates with disease recurrence and overall survival in patients with triple negative breast cancer. Oncotarget, 2016. 7 (47): p. 76628-76634.
16. Guttlein, L. N., et al., Predictive Outcomes for HER2-enriched Cancer Using Growth and Metastasis Signatures Driven By SPARC. Mol Cancer Res, 2017. 15 (3): p. 304-316.
17. McQuerry, J. A., et al., Pathway activity profiling of growth factor receptor network and stemness pathways differentiates metaplastic breast cancer histological subtypes. BMC Cancer, 2019. 19 (1): p. 881.
18. Dhanesuan, N., et al., Doxycycline-inducible expression of SPARC Osteonectin BM40 in MDA-MB-231 human breast cancer cells results in growth inhibition. Breast Cancer Res Treat, 2002. 75 (1): p. 73-85.
19. Ma, J., et al., SPARC inhibits breast cancer bone metastasis and may be a clinical therapeutic target. Oncol Lett, 2017. 14 (5): p. 5876-5882.
20. Koblinski, J. E., et al., Endogenous osteonectin SPARC BM-40 expression inhibits MDA-MB-231 breast cancer cell metastasis. Cancer Res, 2005. 65 (16): p. 7370-7.
21. Alcaraz, L. B., et al., A 9-kDa matricellular SPARC fragment released by cathepsin D exhibits pro-tumor activity in the triple-negative breast cancer microenvironment. Theranostics, 2021. 11 (13): p. 6173-6192.
22. Gascard, P. and T. D. Tlsty, Carcinoma-associated fibroblasts: orchestrating the composition of malignancy. Genes Dev, 2016. 30 (9): p. 1002-19.
23. Dotto, G. P., R. A. Weinberg, and A. Ariza, Malignant transformation of mouse primary keratinocytes by Harvey sarcoma virus and its modulation by surrounding normal cells. Proc Natl Acad Sci USA, 1988. 85 (17): p. 6389-93.
24. Toullec, A., et al., Oxidative stress promotes myofibroblast differentiation and tumour spreading. EMBO Mol Med, 2010. 2 (6): p. 211-30.
25. Benyahia, Z., et al., Stromal fibroblasts present in breast carcinomas promote tumor growth and angiogenesis through adrenomedullin secretion. Oncotarget, 2017. 8 (9): p. 15744-15762.
26. Costa, A., et al., Fibroblast Heterogeneity and Immunosuppressive Environment in Human Breast Cancer. Cancer Cell, 2018. 33 (3): p. 463-479 e10.
27. Wu, S. Z., et al., Stromal cell diversity associated with immune evasion in human triple-negative breast cancer. EMBO J, 2020. 39 (19): p. e104063.
28. Karaayvaz, M., et al., Unravelling subclonal heterogeneity and aggressive disease states in TNBC through single-cell RNA-seq. Nat Commun, 2018. 9 (1): p. 3588.
29. Kieffer, Y., et al., Single-Cell Analysis Reveals Fibroblast Clusters Linked to Immunotherapy Resistance in Cancer. Cancer Discov, 2020. 10 (9): p. 1330-1351.
30. Tariq, M., et al., Macrophage Polarization: Anti-Cancer Strategies to Target Tumor-Associated Macrophage in Breast Cancer. J Cell Biochem, 2017. 118 (9): p. 2484-2501.
31. Fuxe, J. and M.C. Karlsson, TGF-beta-induced epithelial-mesenchymal transition: a link between cancer and inflammation. Semin Cancer Biol, 2012. 22 (5-6): p. 455-61.
32. Jiang, K., et al., Unraveling Heterogeneity of Tumor Cells and Microenvironment and Its Clinical Implications for Triple Negative Breast Cancer. Front Oncol, 2021. 11: p. 557477.
33. Loi, S., et al., Tumor infiltrating lymphocytes are prognostic in triple negative breast cancer and predictive for trastuzumab benefit in early breast cancer: results from the FinHER trial. Ann Oncol, 2014. 25 (8): p. 1544-50.
34. Loi, S., et al., Prognostic and predictive value of tumor-infiltrating lymphocytes in a phase III randomized adjuvant breast cancer trial in node-positive breast cancer comparing the addition of docetaxel to doxorubicin with doxorubicin-based chemotherapy: BIG 02-98. J Clin Oncol, 2013. 31 (7): p. 860-7.
35. Denkert, C., et al., Tumour-infiltrating lymphocytes and prognosis in different subtypes of breast cancer: a pooled analysis of 3771 patients treated with neoadjuvant therapy. Lancet Oncol, 2018. 19 (1): p. 40-50.
36. Dong, H., et al., Tumor-associated B7-H1 promotes T-cell apoptosis: a potential mechanism of immune evasion. Nat Med, 2002. 8 (8): p. 793-800.
37. Miglietta, F., et al., Programmed Cell Death Ligand 1 in Breast Cancer: Technical Aspects, Prognostic Implications, and Predictive Value. Oncologist, 2019. 24 (11): p. e1055-e1069.
38. du Manoir, S., et al., Breast tumor PDXs are genetically plastic and correspond to a subset of aggressive cancers prone to relapse. Mol Oncol, 2014. 8 (2): p. 431-43.
39. Saadoun, H., et al., Prognostic impact of the inclusion of uPA/PAI-1 tumor levels in the current adjuvant treatment decision-making for early breast cancer. Future Oncol, 2014. 10 (2): p. 195-209.
40. Berchem, G., et al., Cathepsin-D affects multiple tumor progression steps in vivo: proliferation, angiogenesis and apoptosis. Oncogene, 2002. 21 (38): p. 5951-5.
41. Laurent-Matha, V., et al., Catalytically inactive human cathepsin D triggers fibroblast invasive growth. J Cell Biol, 2005. 168 (3): p. 489-99.
42. Stuart, T., et al., Comprehensive Integration of Single-Cell Data. Cell, 2019. 177 (7): p. 1888-1902 e21.
43. Goodwin, A. E. and B. U. Pauli, A new adhesion assay using buoyancy to remove non-adherent cells. J Immunol Methods, 1995. 187 (2): p. 213-9.
44. Oo, K. K., et al., Development of an engineered peptide antagonist against periostin to overcome doxorubicin resistance in breast cancer. BMC Cancer, 2021. 21 (1): p. 65.
45. Lindner, J. L., et al., Expression of secreted protein acidic and rich in cysteine (SPARC) in breast cancer and response to neoadjuvant chemotherapy. Ann Oncol, 2015. 26 (1): p. 95-100.
46. Park, S. Y., et al., Heterogeneity for stem cell-related markers according to tumor subtype and histologic stage in breast cancer. Clin Cancer Res, 2010. 16 (3): p. 876-87.
47. Witkiewicz, A. K., et al., Stromal CD10 and SPARC expression in ductal carcinoma in situ (DCIS) patients predicts disease recurrence. Cancer Biol Ther, 2010. 10 (4): p. 391-6.
48. Azim, H. A., Jr., et al., Association between SPARC mRNA expression, prognosis and response to neoadjuvant chemotherapy in early breast cancer: a pooled in-silico analysis. PLOS One, 2013. 8 (4): p. e62451.
49. Gyorffy, B., et al., An online survival analysis tool to rapidly assess the effect of 22,277 genes on breast cancer prognosis using microarray data of 1,809 patients. Breast Cancer Res Treat, 2010. 123 (3): p. 725-31.
50. Nagai, M. A., et al., Prognostic value of NDRG1 and SPARC protein expression in breast cancer patients. Breast Cancer Res Treat, 2011. 126 (1): p. 1-14.
51. Takai, K., et al., Targeting the cancer-associated fibroblasts as a treatment in triple-negative breast cancer. Oncotarget, 2016. 7 (50): p. 82889-82901.
52. Tseng, C. and M. G. Kolonin, Proteolytic Isoforms of SPARC Induce Adipose Stromal Cell Mobilization in Obesity. Stem Cells, 2016. 34 (1): p. 174-90.
53. Sasaki, T., et al., Limited cleavage of extracellular matrix protein BM-40 by matrix metalloproteinases increases its affinity for collagens. J Biol Chem, 1997. 272 (14): p. 9237-43.
54. Sage, E. H., et al., Cleavage of the matricellular protein SPARC by matrix metalloproteinase 3 produces polypeptides that influence angiogenesis. J Biol Chem, 2003. 278 (39): p. 37849-57.
55. Kehlet, S. N., et al., A fragment of SPARC reflecting increased collagen affinity shows pathological relevance in lung cancer-implications of a new collagen chaperone function of SPARC. Cancer Biol Ther, 2018. 19 (10): p. 904-912.
56. Podgorski, I., et al., Bone marrow-derived cathepsin K cleaves SPARC in bone metastasis. Am J Pathol, 2009. 175 (3): p. 1255-69.
57. Pelon, F., et al., Cancer-associated fibroblast heterogeneity in axillary lymph nodes drives metastases in breast cancer through complementary mechanisms. Nat Commun, 2020. 11 (1): p. 404.
58. Straussman, R., et al., Tumour micro-environment elicits innate resistance to RAF inhibitors through HGF secretion. Nature, 2012. 487 (7408): p. 500-4.
59. Feig, C., et al., Targeting CXCL12 from FAP-expressing carcinoma-associated fibroblasts synergizes with anti-PD-L1 immunotherapy in pancreatic cancer. Proc Natl Acad Sci USA, 2013. 110 (50): p. 20212-7.
60. Mhaidly, R. and F. Mechta-Grigoriou, Role of cancer-associated fibroblast subpopulations in immune infiltration, as a new means of treatment in cancer. Immunol Rev, 2021. 302 (1): p. 259-272.

1. A method for predicting the survival time of a subject suffering from triple-negative breast cancer (TNBC) and treating the subject, comprising determining the expression level of Secreted Protein Acidic and Rich in Cysteine (SPARC) in cancer-associated fibroblasts (CAFs) in a biological sample obtained from the subject and treating a subject identified as having an expression level of SPARC in CAFs that is higher than a corresponding reference value with an anti-SPARC-targeted therapy.

2. The method according to claim 1 comprising the steps of i) quantifying the expression of SPARC in CAFs in a biological sample obtained from the subject; ii) comparing the expression quantified at step i) with its predetermined reference value and iii) concluding that the subject will have a short survival time when the expression of SPARC in CAFs is higher than its predetermined reference value or concluding that the subject will have a long survival time when the expression of SPARC in CAFs is lower than its predetermined reference value.

3. The method according to claim 1 wherein the expression of SPARC is determined by immunohistochemistry (IHC).

4. The method according to claim 1 wherein the biological sample is a tumor tissue sample.

5. (canceled)

6. The method according to claim 1, wherein the anti-SPARC-targeted therapy is selected from the group consisting of: a small organic molecule, a polypeptide, an aptamer, an antibody, siRNAs, RNAi, a ribozyme and an antisense oligonucleotide.

7. The method of claim 1, further comprising administering simultaneously, separately or sequentially an anti-PD1 agent with the anti-SPARC-targeted therapy.

8. The method according to claim 7 wherein the anti-PD1 agent is Nivolumab or Pembrolizumab.

9. The method of claim 1, further comprising administering simultaneously, separately or sequentially an anti-PDL1 agent with the anti-SPARC-targeted therapy.

10. The method according to claim 9 wherein the anti-PDL1 agent is selected from the group consisting of: Atezolizumab, Durvalumab, Avelumab and BMS-936559.

11. A kit for use in the method according to claim 1 comprising a reagent that specifically reacts with SPARC mRNA or protein in CAFs and instructions use.

12. A method of screening a drug suitable for the treatment of TNBC comprising i) providing a test compound and ii) determining the ability of said test compound to inhibit the expression and/or activity of SPARC in CAFs.
